(19) United States
(12) Patent Application Publication
(10) Pub. No.: US 2008/0071381 A1
(43) Pub. Date: Mar. 20, 2008

(54) Implant

(76) Inventors: Robin Buscher, Monkeberg (DE); Markus A. Wimmer, Chicago, IL (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/632,535

(22) PCT Filed: Jul. 16, 2004
(86) PCT No.: PCT/EP04/07929

§ 371 Date: Oct. 29, 2007

(51) Int. Cl. A61F 2/30; A61F 2/32; A61F 2/62

(52) U.S. Cl. 623/18.11; 623/22.11; 623/39

(57) ABSTRACT

An implant and method for producing an implant are disclosed. The implant forms a joint with a micro-rough bearing surface formed by a sintered portion. Thus, better wear and friction properties can be achieved.

### CROSS REFERENCES TO RELATED APPLICATIONS

This application is a National Stage filing of International Application PCT/EP2004/007929, filed Jul. 16, 2004, entitled “IMPLANT” This reference is expressly incorporated by reference herein, in its entirety.

### BACKGROUND OF THE INVENTION

1. Field of the Invention

The present invention relates to an implant forming a joint, such as a hip or knee joint, with a bearing surface for a smooth counter-bearing surface, and to a method for producing such an implant.

2. Description of Related Art

The invention is directed towards a reduced emission of wear debris from implants forming a joint and, thus, comprising a bearing surface. Wear debris is of concern because it causes, inter alia, osteolysis and may lead to undesired effects in a body in which the implant is implanted.

So-called metal-on-metal prostheses are currently manufactured with a minimum surface roughness, in order to minimize debris. However, such a hip implant usually has a wear of about 30 μm during the starting phase of use (“running in”) and of about 5 μm in the following years. The number of the emitted particles (debris) is about 10¹⁰ to 10¹⁵ per year.

WO 03/044383 A1 and the articles “Sliding Wear Behavior of an Electrochemically Modified Austenitic Nitrogen Steel Surface” of Büscher et al, Wear 255 Issue 12 (2003), page 1318-1325 and “Sliding Wear Behavior of Wet-Chemically Modified High-Nitrogen Steels” of Büscher et al, Lubricants, Materials and Lubrication Engineering (Proc Conf) 13th Int. coll. Triboloci, January 15-17 T A Esslingen, Germany (2002), page 1297-1307, disclose an implant with a bearing surface roughened by etching in order to minimize build-up of debris and to reduce friction. The etching results in that the surface shape or dimensions are altered so that it is very difficult to achieve a desired, pre-determined geometry of the bearing surface with high accuracy.

In chapter 9 of the book “Handbook of Materials for Medical Devices”, published by ASM International, ISBN: 0-87170-700-X, porous coatings for orthopedic implants are disclosed. The porous coatings may be produced by sintering spherical metal powders. However, these coatings are used for better fixation of the implants in that bone can grow into the porous coating. Therefore, the pore size shall range from 100 to 500 μm. The bearing surface is formed e.g. by a forged Cr-femoral head, i.e. the porous coating is not used for forming a bearing surface.

U.S. Pat. No. 6,576,014 B2 discloses an orthopedic implant, wherein an elongated stem comprises a porous layer made of sintered metal particles to enhance bone ingrowth or the mechanical interlock with bone cement. The porous layer is not used for forming a bearing surface.

U.S. Pat. No. 5,308,412 A discloses an orthopedic implant made of a cobalt-chromium or cobalt-chromium-molybdenum alloy. The implant is exposed to molecular nitrogen gas or ionized nitrogen at a process temperature and for a process time duration sufficient to enhance surface hardness and wear resistance properties, but without the formation of a measurable nitrogen layer that tends to increase surface roughness and brittleness and diminished wear resistance properties. Thus, a rough bearing surface is avoided. Instead, a smooth and hard bearing surface is preferred.

M. A. Wimmer et. al. describe in “The acting wear mechanisms on metal-on-metal hip joint bearings: in vitro results”, Wear 250 (2001), p 129-139, and “Tribochemical Reaction on Metal-On-Metal Hip Joint Bearings—A Comparison between in-vitro and in-vivo Results”, Wear 255 (2003), p 1007-1014, that layers of denatured or decomposed proteins can be formed on metal-on-metal hip joint bearing surfaces. These layers can reduce wear and friction.

There is a need to provide an implant and a method for producing an implant, wherein a roughened bearing surface with desired properties can be easily achieved with high accuracy.

### BRIEF SUMMARY

An object of the present invention is to provide an implant with a micro-rough bearing surface and a method for producing such an implant, wherein the implant can be produced easily with high accuracy and/or with the desired properties so that wear and the generation of debris can be minimized.

The above object is achieved by an implant according to the present invention or by a method according to the present invention. Preferred embodiments are also described herein.

One aspect of the present invention is to provide a portion made by sintering of particles together for forming a micro-rough bearing surface. Thus, it is possible to produce the implant with the desired properties with high accuracy.

The terms “sintered” or “sinter” are to be understood, here, as meaning in the usual sense that a coherent bonded mass is formed by heating metal powders or particles without melting, in particular so-called powder metallurgy. In a broader sense, these terms also include the application of high isostatic pressure forming the coherent mass or body from the powder or particles.

“Micro-rough” is to be understood here as meaning that the surface is made to have a rough form—preferably into the μm range—in such a way that particles, preferably of up to 10 μm or even up to 100 μm, can be at least partly accepted by depressions or cavities with openings to the surface, and in particular be embedded or entrapped in the depressions or cavities.

The micro-rough formation of the bearing surface leads to several advantages:
Firstly, debris or particles occurring during use of the joint formed by the implant can be accepted in the cavities of depressions, and in particular be permanently entrapped in them. This applies in particular to nano- or microsize-particles, which primarily occur when two metallic surfaces slide on each other. In this way, wear can be effectively reduced or even minimized.
Secondly, the micro-rough bearing surface can be adapted more easily to an assigned counter-bearing surface. This is made possible in particular by plastic deformation or flattening of micro-bumps or the like. In this way, the bearing preferably formed as a sliding bearing or joint, “runs in” more quickly and/or with less debris.
Thirdly, the depressions of the micro-rough bearing surface can form a lubricant reservoir. This is conducive to reducing the friction and/or increasing the service life.
Fourthly, the micro-rough bearing surface is enlarged significantly in its surface area in comparison with a smooth surface. The enlarged surface area is better able to bind or retain particles and/or lubricant. This is in turn conducive to reducing the friction and/or prolonging the service life, in particular by reducing the three-body abrasion caused by free particles.
Fifthly, the micro-roughness of the bearing surface can enhance the formation of decomposed or denatured proteins and/or the formation or adhesion of a layer of such proteins when using the joint/bearing in the human body. This is in turn conducive to reducing the friction and/or prolonging the service life.

The micro-rough bearing surface is preferably made at least for the most part, in particular entirely, to have a macroscopically smooth shape; consequently, the bearing surface appears to the human eye to be smooth, even if colorations or optical effects may possibly give the bearing surface an appearance of varying color.

Further aspects, advantages, properties and features of the present invention are explained in more detail below on the basis of the drawings.

### BRIEF DESCRIPTION OF THE SEVERAL VIEWS OF THE DRAWINGS

FIG. 1 shows a proposed implant, formed as a hip joint and including a micro-rough bearing surface.

FIG. 2 shows a schematic sectional representation of an enlarged detail of the bearing surface, ignoring the curvature of the bearing surface existing in the case of the embodiment according to FIG. 1.

FIG. 3 shows a schematic representation similar to FIG. 2 of a modified embodiment.

### DETAILED DESCRIPTION

For the purposes of promoting an understanding of the disclosure, reference will now be made to the embodiments illustrated in the drawings and specific language will be used to describe the same. It will nevertheless be understood that no limitation of the scope of the disclosure is thereby intended, such alterations and further modifications in the illustrated device and its use, and such further applications of the principles of the disclosure as illustrated therein being contemplated as would normally occur to one skilled in the art to which the disclosure relates.

In the figures, the same designations are used for identical or similar parts, corresponding or comparable advantages and properties being achieved even if the description is not repeated for reasons of simplification.

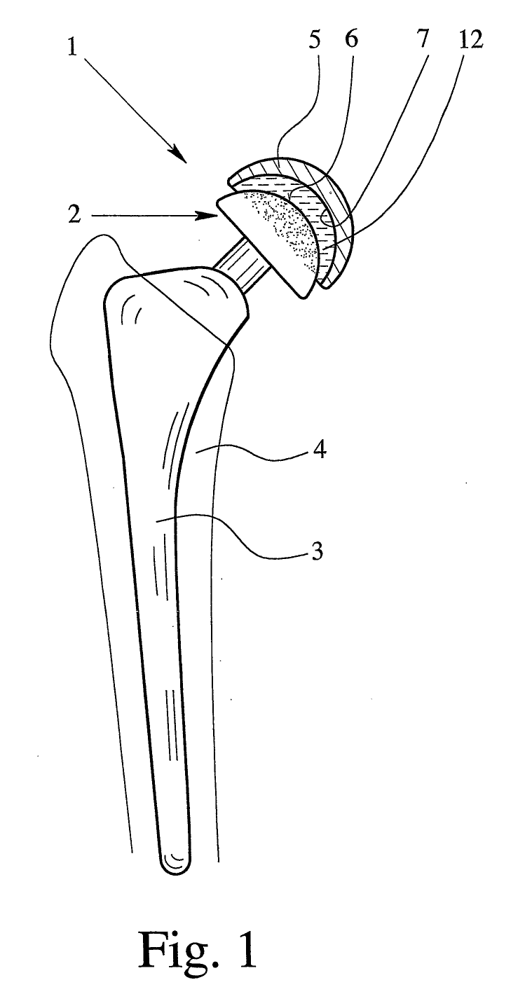

FIG. 1 shows in a schematic representation an embodiment of an orthopedic implant 1 according to the present invention. In case of the example represented, the implant 1 forms a joint, namely a hip joint, i.e. an artificial hip for a patient (not illustrated). However, it may for example also be some other joint, such as an artificial knee joint, or some other implant performing a bearing function, or some other prosthesis with a joint, or any other artificial joint in the human body.

The implant 1 according to FIG. 1 comprises a bearing head 2 connected to a stem 3. In the implantation, the stem 3 is inserted into a femur 4, indicated in FIG. 1, and a bearing shell or cup 5 associated to the bearing head 2 is inserted into an assigned region of the hip bone (not illustrated).

The bearing head 2 and the assigned bearing cup 5 are illustrated in FIG. 1 in a state in which they have been moved apart from each other for illustrative reasons. For purposes of illustration, the bearing cup 5 is represented in section.

The bearing head 2 and the assigned bearing cup 5 forms a joint or bearing, in particular a sliding bearing. It may, however, also be some other bearing, such as a roller or rolling bearing.

Instead of the formation as the bearing head 2 and bearing cup 5, the bearing elements assigned to each other may also have some other form, adapted to the respectively intended use.

The bearing head 2 according to FIG. 1 forms a bearing surface 6 cooperating with the assigned bearing cup 5. This bearing surface 6 is made micro-rough at least partially and for example located as dotted for purposes of illustration in FIG. 1.

In fact, the roughening of the bearing surface 6 in the dotted area or in the entire area is formed so finely that the bearing surface 6 usually appears to be smooth to the human eye, even if the roughening gives the bearing surface 6 in the micro-rough region the appearance of varying color.

The bearing surface 6 is assigned a counter-bearing surface 7, which is formed by the bearing cup 5. In the present embodiment, the counter-bearing surface 7 is formed such that it complements the bearing surface 6. However, the counter-bearing surface 7 may—depending on the intended use and bearing structure—also have a form deviating from the complementary surface form. This applies in particular to other sliding bearings, roller or rolling bearing.

In the present case, the bearing surface 6 and the counter-bearing surface 7 slide on each other, that is to say, form a sliding bearing. However, rolling movements may also be superimposed on the sliding movement. As already mentioned above, other forms of bearings may, in principle, also be realized, for example with a planar bearing surface 6 and/or counter-bearing surface 7 or with primarily rolling movement.

The counter-bearing surface 7 is preferably made at least substantially to have a smooth form, that is to say, preferably both macroscopically smooth and microscopically/nanoscopically smooth, (i.e. not micro-rough). Preferably, the counter-bearing surface 7 is at least as hard as the bearing surface 6 or harder.

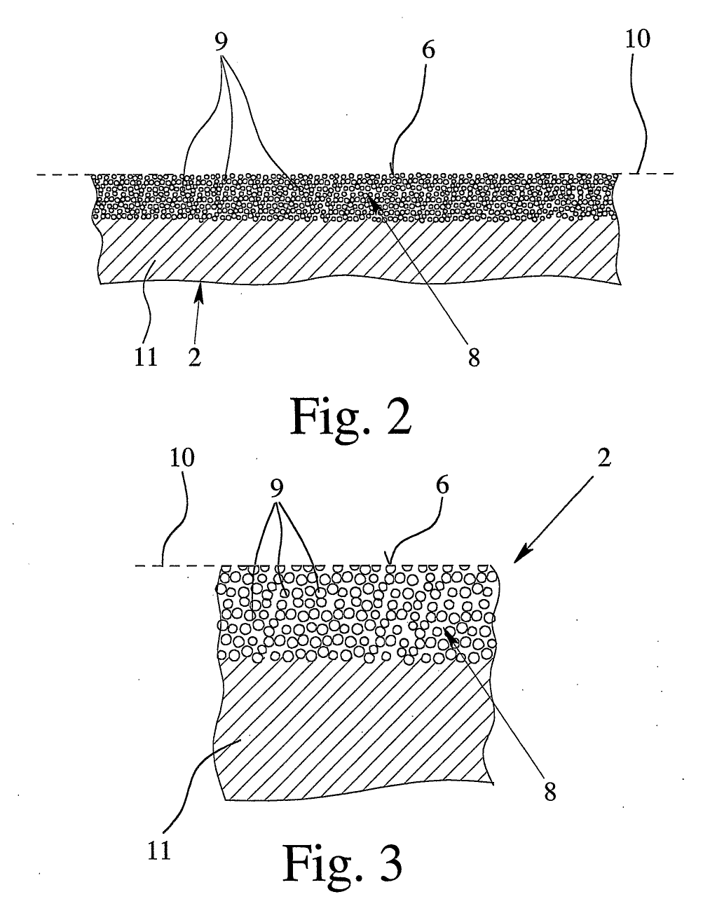

In the following, the formation of the micro-rough bearing surface 6 will be explained with reference to FIG. 2. However, it has to be noted that the counter-bearing surface 7 could be made micro-rough instead of bearing surface 6 in a similar manner as described in the following as an alternative.

FIG. 2 shows in an enlarged section, a part of the bearing head 2 forming the micro-rough bearing surface 6. The macroscopic curvature of the bearing surface 6, that is the spherical-head-like or dome-like formation of the bearing surface 6 has been omitted in FIG. 2 in order to simplify the illustration. Instead of this, the bearing surface 6 is represented as macroscopically planar in FIG. 2.

In order to achieve the nano- or micro-structuring of the bearing surface 6 according to the present invention, the implant 1 comprises a portion 8 formed by sintering of preferably spherical particles 9 together, in particular of powder or the like. The sintering has to be understood in the sense as described above. In addition, chapter 9 of the book “Handbook of Materials for Medical Devices”, published by ASM International, ISBN: 0-87170-700-X, and U.S. Pat. No. 6,576,014 B2 are incorporated herewith as reference regarding the possibilities for sintering the particles 9 together and for forming the portion 8. The portion 8 is hereinafter called sintered portion 8 due to its formation.

The sintered portion 8 comprises preferably irregular depressions, cavities and/or openings to its surface 6 and, thus, forms the micro-rough bearing surface 6. In particular, debris or loose particles 9 or other particles can be embedded and entrapped in depressions or cavities of the sintered portion 8 opening to the bearing surface 6. Similarly, the actual surface of the micro-rough bearing surface 6 is significantly larger than the macroscopic area of the extent of the bearing surface 6 provided by its macroscopically smooth contour 10.

The macroscopically smooth contour 10 may be regarded as the intended profile of the bearing surface 6, desired in the case of macroscopically customary machining, for example by cutting, grinding or polishing, which is preferably macroscopically smooth.

The averaged roughness represents the average deviation of elevations and depressions from the average, macroscopically smooth intended surface or contour 10. Preferably, the averaged roughness of the bearing surface 6 is at least 1 μm, in particular at least 5 μm or 10 μm, and/or at most 200 or 100 μm, in particular up to 50 μm or lower.

The peak-to-valley height, i.e. the maximum difference in height between one of the elevations and one of the depressions in the micro-rough bearing surface 6, is preferably at most 200 μm, in particular of 100 μm or less.

The desired micro-roughness of the bearing surface 6 is preferably achieved as described in the following.

The particles 9 are preferably made of metal or a metal alloy or any other biocompatible material. In particular, the particles 9 are made of a cobalt-based alloy, a cobalt-chromium-molybdenum alloy, a titanium alloy, e.g. TiAl6V4, a tantalum alloy and/or stainless steel. The particles 9 can consist of any one of the materials named in U.S. Pat. No. 6,576,014 B2 or U.S. Pat. No. 5,308,412 A, which are incorporated herewith by reference.

Most preferably, the particles consist of 40 to 60% by weight of cobalt (Co), 26 to 30% by weight of chromium (Cr), 5 to 7% by weight of molybdenum (Mo), and the rest of additives.

The particles 9 are powder-like and are also called beads. The average diameter of the particles 9 is preferably 0.5 to 1000 μm.

The particles 9 are bonded together by sintering in the above sense, including the possibility of hot isostatic pressing (HIP).

For sintering, the particles 9 are subjected e.g. to a temperature of about 950° C. to 1150° C. for 1 to 3 hours in an inert atmosphere. During hot isostatic pressing, the particles 9 are subjected e.g. to a temperature of about 900° C. to 1100° C. and to a pressure of about 100 to 150 MPa for 1 to 3 hours in an inert atmosphere.

The heating energy can be supplied by an oven, by laser, by an electron beam, by microwaves, by a plasma or the like.

In the preferred embodiment, the sintered portion 8 consisting of the particles 9 forms a covering layer on a substrate 11 of the implant 1 and the bearing head 2, respectively. This covering layer has a thickness of preferably about 10 to 1000 μm, in particular of 50 to 500 μm.

The sintered portion 8 is porous, i.e. forms a porous coating in the embodiment shown in FIG. 2.

The sintered portion 8 has a porosity of at least 5%, preferably at least 10% or more at the micro-rough bearing surface 6. This porosity value reflects the area ratio of the free area, i.e. the areas not covered by the particles 9 in the top layer of particles at the surface, to the total area.

The sintered portion 8 has preferably a porosity of at most 35% at the micro-rough bearing surface 6.

The sintered portion 8 has preferably a bulk porosity of at least 5 vol.-%, preferably of 10 vol.-% or more, and/or of at most 35 vol.-%. This bulk porosity value indicates the spatial ratio of air in a volume of the covering layer formed by the sintered portion 8.

Due to the preparation of the sintered portion 8 as described above, the sintered portion 8 comprises a plurality of irregular depressions, cavities and openings towards the bearing surface 6 and, thus, result in the desired micro-roughness.

The average surface density of depressions, cavities or its openings to the bearing surface 6 is preferably at least 10/mm², in particular at least 1·10²/mm², 1·10³/mm² or 1·10⁴/mm².

Depending on the application or design of the implant 1, the sintered portion 8 may also be larger than the bearing surface 6, i.e. may extend beyond the (required) bearing surface 6, e.g. cover the stem 3 or the complete implant 1, in particular to enhance bone ingrowth or the mechanical interlock with bone cement or the like in these other regions.

As already indicated, the micro-rough bearing surface 6 and, thus, the sintered portion 8 may alternatively or additionally be formed at the bearing cup 5 or form the counter-bearing surface 7.

Further, the bearing head 2 and/or bearing cup 5 may comprise smooth bearing portions in addition to the micro-rough bearing surface 6.

Furthermore, the sintered portion 8 or a porous coating with similar properties can also be made as described in chapter 9 of the book “Handbook of Materials for Medical Devices”, published by ASM International, ISBN: 0-87170-700-X, or as described in U.S. Pat. No. 6,576,014 B2, which are incorporated herein by reference.

According to a preferred embodiment, the sintered portion 8 and, thus, the bearing surface 6 are made only by sintering, i.e. no further machining or the like is necessary. This is possible due to the high dimensional accuracy that can be achieved with sintering.

Alternatively, it is provided that the sintered portion 8 exceeds the desired contour 10 and, thus, is machined in order to obtain the bearing surface 6 with the desired dimensions, i.e. the desired contour 10. Preferably, the sintered portion 8 is cut and/or grinded and, then, polished. FIG. 3 shows in a schematic illustration the resulting bearing surface 6 with flattened particles 9 in the top layer at the surface.

In both embodiments according to FIG. 2 and FIG. 3, the contour 10 may be curved or have any other suitable form as desired.

In both embodiments according to FIG. 1 and 2, the sintered portion 8 forms a coating or layer, i.e. is formed on the substrate 11. Preferably, the substrate 11 is massive and/or forged or cast. Preferably, it is made of the same material as the particles 9 or of a similar material with at least basically similar electrochemical negativity in order to avoid electrochemical reactions in the human body in the implanted state.

Alternatively, the substrate 11 can be made of plastic, ceramic, metal, metal oxide, or any combination or composite thereof. In particular, the substrate 11 can be made of any one of the materials named in U.S. Pat. No. 6,576,014 B2 or U.S. Pat. No. 5,308,412 A, which are incorporated herewith by reference.

According to a further alternative, the substrate 11 can also be made of sintered material, as well, preferably with another, in particular lower porosity than the sintered portion 8 forming the covering layer in order to achieve the desired strength and mechanical properties of the implant 1.

As already described, the counter-bearing surface 7 is preferably smooth. However, if desired, the counter-bearing surface 7 may also be made to have at least in a certain region or regions a micro-rough form. According to a design variant, the counter-bearing surface 7 is provided with fine outwardly open pores or cavities, for example with an average diameter of 100 nm to 20 μm.

In any case, the counter-bearing surface 7 is formed from a suitable material, such as plastic, ceramic, metal oxide, metal or any combination or composite thereof. It can be a coating or layer, e.g. of diamond like carbon (DLC) or any other suitable material.

The counter-bearing surface 7 is preferably harder than the bearing surface 6 in order to achieve the desired embedding of particles/debris in the open cavities or depressions of the bearing surface 6.

In particular, the counter-bearing surface 7 is formed from metal. Preferably, the counter-bearing surface 7 can be made of any one of the materials named in U.S. Pat. No. 6,576,014 B2 or U.S. Pat. No. 5,308,412 A, which are incorporated herewith by reference. However, the counter-bearing surface 7 may, for example, also be formed from a dissimilar material as the bearing surface 6.

According to one alternative, the counter-bearing surface 7 or bearing cup 5 is forged or cast.

Preferably, the (metallic) micro-rough bearing surface 6 is combined with a metal counter-bearing surface 7, in particular of cobalt, a cobalt alloy, steel, a cobalt-chromium-molybdenum alloy, a tantalum alloy, or any other suitable biocompatible metal or alloy so that a metal-on-metal bearing is formed.

However, the metal bearing surface 6 can also be combined with the counter-bearing surface 7 made of ceramic, like Al₂O₃, or plastic, like ultra high molecular weight ethylene.

If the counter-bearing surface 7 is made of plastic, such as polyethylene, the bearing surface 6 should be formed or polished as shown in FIG. 3 and/or is preferably made of ceramic, such as aluminum oxide, or of metal, such as steel or an alloy based on chromium, titanium or tantalum.

In the case of the example represented, the micro-rough bearing surface 6 is formed on the bearing head 2 and the counter-bearing surface 7 is formed on the bearing cup 5. However, this may also be reversed.

Depending on use, the bearing surface 6 and the counter-bearing surface 7 may slide directly on each other, that is to say possibly form a lubricant-free bearing. However, the bearing surface 6 can assimilate body fluids and/or decomposed or denatured proteins as lubricant 12, as indicated in FIG. 1. In particular, the micro-roughness of the bearing surface 6 and/or counter-bearing surface 7 may enhance the formation of decomposed or denatured proteins and/or the adhesion of a layer of such proteins on the micro-rough surfaces when using the joint/bearing in the human body. This kind of proteins and this effect are described by M. A. Wimmer et. al. in “The acting wear mechanisms on metal-on-metal hip joint bearings: in vitro results”, Wear 250 (2001), p 129-139, and “Tribochemical Reaction on Metal-On-Metal Hip Joint Bearings—A Comparison between in-vitro and in-vivo Results”, Wear 255 (2003), p 1007-1014, which articles are incorporated herewith by reference.

The proposed micro-rough formation of the bearing surface 6, in particular in conjunction with a preferably at least substantially smooth and/or harder counter-bearing surface 7, leads to the effect that very quick running-in is made possible, with low particle formation or at least low particle shedding. Moreover, relatively low friction is obtained. This can be explained by the fact that a rapid adaptation of the bearing surface 6, preferably if formed from a tough and/or ductile material, in particular metal, to the counter-bearing surface 7 takes place in the running-in phase, it being possible for loose particles/debris that may otherwise lead to undesired three-body abrasion to be accepted by the cavities or depressions of the bearing surface 6. Moreover, the lubricant 12 adheres particularly well on the large surface area 9 of the bearing surface 4, a relatively large lubricant reservoir also forming in the depressions 7, so that low friction, in particular sliding friction, is made possible.

Tests have shown that a further advantageous effect can occur. In particular in the case of metallic bearing surfaces 6, the metal particles 9 or debris can—at least in a certain region or regions—form a very solid particle layer, of for example approximately 10 to 100 nm in thickness, on the elevations of the bearing surface 6. A high strength of the particle layer can be obtained in particular for the reason that, on account of their small size, the individual metal particles 9 oxidize at least partially, in particular at least largely completely. A particularly hard layer, which is accordingly very wear-resistant or abrasion-resistant, then forms from the at least partially oxidized and/or ceramic-like particles 9.

Preferably, the implant 1 is used in such a way that the nominal surface pressure of the bearing surface 6 is at most 100 MPa, in particular at most 50 MPa or 20 MPa.

High pressure and/or temperatures at each or some of the elevations of the bearing surface 6 may lead to denaturing of proteins of the human body at these elevations and, thus, to an additional reduction in wear and/or friction.

If desired, further surface parts of the implant 1, e.g. at least part of the stem 3, can be provided with the sintered portion 8 as a porous coating or layer in order to achieve an improved fixation in the bone, in the embodiment according to FIG. 1 in the femur 4. If desired, the implant 1 or the bearing head 2 with its associated stem 3 can be coated completely with the porous sintered portion 8.

Additionally or alternatively to the sintered portion 8, the micro-roughness of the bearing surface 6 can also be achieved by laser machining, preferably laser ablation, of a smooth surface, in particular with the same or similar properties and/or dimensions as by sintering.

### INDUSTRIAL APPLICABILITY

The present invention is, in particular, useful for orthopedic implants 1, like artificial hips or knee joints, or for any other artificial joints in the human body. There are approximately 200.000 primary hip operations each year in the U.S. alone and an estimated number of 800.000 worldwide. The micro-structured features allow solid lubrication and wear debris entrapment and, thus, increase the service life and the properties of implants with joints according to the present invention.

While the preferred embodiment of the invention has been illustrated and described in the drawings and foregoing description, the same is to be considered as illustrative and not restrictive in character, it being understood that all changes and modifications that come within the spirit of the invention are desired to be protected.

1-54. (canceled)

55. An implant forming a joint, comprising a sintered portion forming a micro-rough bearing surface for a smooth counter-bearing surface, said sintered portion having a porosity of at least 5% at said micro-rough bearing surface.

56. The implant according to claim 55, wherein said sintered portion has a porosity of at least 10% at said micro-rough bearing surface.

57. The implant according to claim 55, wherein said sintered portion has a porosity of at most 35% at said micro-rough bearing surface.

58. The implant according to claim 55, wherein said sintered portion has a bulk porosity of at least 5 vol.-%, preferably of 10 vol.-% or more.

59. The implant according to claim 55, wherein said sintered portion has a bulk porosity of at most 35 vol.-%.

60. The implant according to claim 55, wherein said sintered portion is made of preferably sphere-like particles.

61. The implant according to claim 60, wherein said particles have a mean diameter of 0.5 to 100 μm.

62. The implant according to claim 60, wherein said particles are made of metal or a metal alloy.

63. The implant according to claim 55, wherein said particles are made of a Co-based-alloy, a Co—Cr—Mo-alloy, a titanium alloy, a tantalum alloy or stainless steel.

64. The implant according to claim 60, wherein said particles consist of 40 to 70% by weight of Co, 26 to 30% by weight of Cr, 5 to 7% by weight of Mo, and the rest of additives.

65. The implant according to claim 55, wherein said bearing surfce is made to have a macroscopically smooth form.

66. The implant according to claim 55, wherein said bearing surface is finally formed by sintering, grinding and/or polishing.

67. The implant according to claim 55, wherein said sintered portion comprises irregular depressions, cavities and/or openings.

68. The implant according to claim 55, wherein said bearing surface has an averaged roughness or a peak-to-valley height of at most 200 μm, in particular up to 100 μm or 50 μm.

69. The implant according to claim 55, wherein said bearing surface has at least substantially no microscopically planar surface portions.

70. The implant according to claim 55, wherein said sintered portion forms a covering layer of said implant.

71. The implant according to claim 70, wherein said covering layer has a thickness of 10 to 1000 μm, preferably 50 to 500 μm.

72. The implant according to claim 55, wherein said sintered portion is supported by a substrate.

73. The implant according to claim 72, wherein said substrate is massive or sintered or a composite structure.

74. The implant according to claim 72, wherein said substrate is made of metal, metal oxide, ceramic, plastic, or any combination or composite thereof.

75. The implant according to claim 55, wherein said implant at least essentially completely consists of sintered material.

76. The implant according to claim 72, wherein said sintered portion has a lower porosity than the rest of said implant.

77. The implant according to claim 55, wherein said bearing surface is assigned the counter-bearing surface.

78. The implant according to claim 72, wherein the counter-bearing surface is harder than said bearing surface.

79. The implant according to claim 77, wherein the counter-bearing surface is made of metal, metal oxide, ceramic plastic, or any combination or composite thereof.

80. The implant according to claim 55, wherein said implant forms a sliding bearing.

81. The implant according to claim 55, wherein said implant forms at least one part of an artificial hip or knee joint.

82. A method for producing an implant with a micro-rough bearing surface, wherein particles are sintered together as a sintered portion for forming said micro-rough bearing surface.

83. The method according to claim 82, wherein said sintered portion has a porosity of at least 5%, preferably at least 10% or more, at said micro-rough bearng surface.

84. The method according to claim 82, wherein said sintered portion has a porosity of at most 35% at said micro-rough bearing surface.

85. The method according to claim 82, wherein said sintered portion has a bulk porosity of at least 5 vol.-%, preferably of 10 vol.-% or more.

86. The method according to claim 82, wherein said sintered portion has a bulk porosity of at most 35 vol.-%.

87. The method according to claim 82, wherein said particles are sphere-like.

88. The method according to claim 82, wherein said particles have a mean diameter of 0.5 to 100 μm.

89. The method according to claim 82, wherein said particles are made of metal or a metal alloy.

90. The method according to claim 82, wherein said particles are made of a Co-based-alloy, a Co—Cr—Mo-alloy, a titanium alloy, a tantalum alloy, or stainless steel.

91. The method according to claim 82, wherein said particles consist of 40 to 70% by weight of Co, 26 to 30% by weight of Cr, 5 to 7% by weight of Mo, and the rest of additives.

92. The method according to claim 82, wherein said bearing surface is made to have a macroscopically smooth form.

93. The method according to claim 82, wherein said bearing surface is finally formed by sintering, grinding and/or polishing.

94. The method according to claim 82, wherein said sintered portion comprises irregular cavities and/or openings.

95. The method according to claim 82, wherein said bearing surface has an averaged roughness or a peak-to-valley height of at most 200 μm, in particular up to 100 μm or 50 μm.

96. The method according to claim 82, wherein said bearing surface has at least substantially no microscopically planar surface portions.

97. The method according to claim 82, wherein said sintered portion forms a covering layer of said implant.

98. The method according to claim 97, wherein said covering layer has a thickness of 10 to 1000 μm, preferably 50 to 500 μm.

99. The method according to claim 82, wherein said sintered portion is formed on a substrate.

100. The method according to claim 99, wherein said substrate is massive or sintered or a composite structure.

101. The method according to claim 99, wherein said substrate is made of metal, metal oxide, ceramic or plastic.

102. The method according to claim 82, wherein said implant at least essentially completely consists of sintered material.

103. The method according to claim 102, wherein said sintered portion has a lower porosity than the rest of said implant.

104. The method according to claim 82, wherein said a preferably smooth counter-bearing surface is located adjacent to said bearing surface.

105. The method according to claim 104, wherein said counter-bearing surface is harder than said bearing surface.

106. The method according to claim 104, wherein said counter-bearing surface is made of metal, metal oxide, ceramic or plastic.

107. The method according to claim 82, wherein said implant forms a sliding bearing.

108. The method according to claim 82, wherein said implant forms at least one part of an artificial hip or knee joint.
